(19) United States
(12) Patent Application Publication
(10) Pub. No.: US 2022/0375567 A1
(43) Pub. Date: Nov. 24, 2022

(54) REHABILITATION PLANNING APPARATUS, REHABILITATION PLANNING SYSTEM, REHABILITATION PLANNING METHOD, AND COMPUTER READABLE MEDIUM

(71) Applicant: NEC Corporation, Minato-ku, Tokyo (JP)

(72) Inventors: Toshinori HOSOI, Tokyo (JP); Yuki KOSAKA, Tokyo (JP); Masahiro KUBO, Tokyo (JP); Yuan LUO, Tokyo (JP); Shigemi KITAHARA, (US)

(73) Assignee: NEC Corporation, Minato-ku, Tokyo (JP)

(21) Appl. No.: 17/761,285

(22) PCT Filed: Jul. 29, 2020
(86) PCT No.: PCT/JP2020/029076

§ 371 Date: Mar. 17, 2022

(30) Foreign Application Priority Data
Oct. 4, 2019 (JP) 2019-184152

(51) Int. Cl. G16H 20/30; G16H 10/60

(52) U.S. Cl. CPC G16H 20/30; G16H 10/60

(57) ABSTRACT

A rehabilitation planning apparatus includes: a similarity calculation unit that calculates a degree of similarity between each of a plurality of pieces of past information and target patient information, each of the plurality of pieces of past information being information about a respective one of a plurality of past patients who performed rehabilitation in a past, and the target patient information being information about a target patient; a ranking unit that extracts at least one piece of past information from the plurality of pieces of past information based on the degree of similarity, and ranks the at least one piece of past information according to a predetermined ranking rule; and a determination unit that determines, as a rehabilitation plan for the target patient, a rehabilitation history of the past patient associated with the past information according to a result of the ranking of the pieces of past information.

### TECHNICAL FIELD

The present invention relates to a rehabilitation planning apparatus, a rehabilitation planning method, and a program.

### BACKGROUND ART

Patent Literature 1 discloses an exercise program presentation system capable of presenting an exercise program corresponding to the symptom of a user and a time at which the effect of the exercise program, when it is performed, will appear.

In recent years, there has been a need for a technology for supporting rehabilitation (e.g., a rehabilitation training or a rehabilitation therapy) (hereinafter also referred to as “rehab”) as described above, and research and development for such technology has been pursued. In general, a patient performs rehabilitation according to a rehabilitation plan prepared in advance.

### CITATION LIST

### Patent Literature

Patent Literature 1: Japanese Unexamined Patent Application Publication No. 2017-27192

### SUMMARY OF INVENTION

### Technical Problem

In general, a rehabilitation plan is created by a therapist such as a physical therapist after some deliberation. However, in such a case, the person who has created the rehabilitation plan needs to examine the plan based on his/her experiences and advice from other therapists. Therefore, it takes time to examine the rehabilitation plan.

One of the objects to be attained by example embodiments disclosed in this specification is to provide a rehabilitation planning apparatus, a rehabilitation planning method, and a program capable of efficiently creating a rehabilitation plan.

### Solution to Problem

A rehabilitation planning apparatus according to a first aspect of the present disclosure includes:

similarity calculation means for calculating a degree of similarity between each of a plurality of pieces of past information and target patient information, each of the plurality of pieces of past information being information about a respective one of a plurality of past patients who performed rehabilitation in a past, and the target patient information being information about a target patient;

ranking means for extracting at least one piece of past information from the plurality of pieces of past information based on the degree of similarity, and ranking the at least one piece of past information according to a predetermined ranking rule; and

determination means for determining, as a rehabilitation plan for the target patient, a rehabilitation history of the past patient associated with the past information according to a result of the ranking of the pieces of past information.

A rehabilitation planning method according to a second aspect of the present disclosure includes:

calculating a degree of similarity between each of a plurality of pieces of past information and target patient information, each of the plurality of pieces of past information being information about a respective one of a plurality of past patients who performed rehabilitation in a past, and the target patient information being information about a target patient;

extracting at least one piece of past information from the plurality of pieces of past information based on the degree of similarity, and ranking the at least one piece of past information according to a predetermined ranking rule; and

determining, as a rehabilitation plan for the target patient, a rehabilitation history of the past patient associated with the past information according to a result of the ranking of the pieces of past information.

A program according to a third aspect of the present disclosure causes a computer to perform:

a similarity calculation step of calculating a degree of similarity between each of a plurality of pieces of past information and target patient information, each of the plurality of pieces of past information being information about a respective one of a plurality of past patients who performed rehabilitation in a past, and the target patient information being information about a target patient;

a ranking step of extracting at least one piece of past information from the plurality of pieces of past information based on the degree of similarity, and ranking the at least one piece of past information according to a predetermined ranking rule; and

a determination step of determining, as a rehabilitation plan for the target patient, a rehabilitation history of the past patient associated with the past information according to a result of the ranking of the pieces of past information.

### Advantageous Effects of Invention

According to the present disclosure, it is possible to provide a rehabilitation planning apparatus, a rehabilitation planning method, and a program capable of efficiently creating a rehabilitation plan.

### BRIEF DESCRIPTION OF DRAWINGS

FIG. 1 is a block diagram showing an example of a configuration of a rehabilitation planning apparatus according to an outline of an example embodiment;

FIG. 2 is a block diagram showing an example of a configuration of a rehabilitation planning system according to a first example embodiment;

FIG. 3 is a schematic diagram showing an example of a rehabilitation plan displayed on a terminal device under the control of an output control unit;

FIG. 4 is a schematic diagram showing an example of a rehabilitation plan displayed on a terminal device under the control of an output control unit;

FIG. 5 is a schematic diagram showing examples of comparisons between ability values displayed on a terminal device under the control of an output control unit;

FIG. 6 is a schematic diagram showing an example of a hardware configuration of a rehabilitation planning apparatus according to the first example embodiment;

FIG. 7 is a flowchart showing an example of operations performed by a rehabilitation planning system according to the first example embodiment;

FIG. 8 is a block diagram showing an example of a configuration of a rehabilitation planning system according to a second example embodiment;

FIG. 9 is a flowchart showing an example of operations performed by the rehabilitation planning system according to the second example embodiment;

FIG. 10 is a block diagram showing an example of a configuration of a rehabilitation planning system according to a third example embodiment; and

FIG. 11 is a flowchart showing an example of operations performed by a rehabilitation planning system according to third example embodiment.

### DESCRIPTION OF EMBODIMENTS

### Overview of Example Embodiment

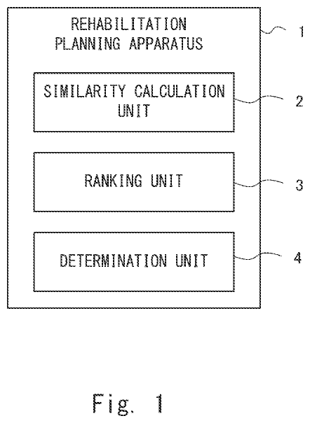

Prior to describing an example embodiment in detail, an outline of the example embodiment will be described. FIG. 1 is a block diagram showing an example of a configuration of a rehabilitation planning apparatus 1 according to an outline of an example embodiment. As shown in FIG. 1, the rehabilitation planning apparatus 1 includes a similarity calculation unit 2, a ranking unit 3, and a determination unit 4.

The similarity calculation unit 2 calculates the degree of similarity between each of a plurality of pieces of past information, which is information about a respective one of a plurality of past patients, and target patient information, which is information about a target patient. Note that the target patient is a patient who is scheduled to perform rehabilitation according to a rehabilitation plan to be created. The past patients are patients who are different from the target patient and performed rehabilitation in the past.

The past information is a set of pieces of information about the past patients, and the target patient information is a set of pieces of information about the target patient. More specifically, the past information is a set of pieces of information representing features of the past patients, and the target patient information is a set of pieces of information representing features of the target patient.

Note that at least some of the items in the past information (types of information included in the past information) correspond to some of the items in the target patient information (types of information included in the target patient information). Therefore, it is possible to compare some items in the past information with the corresponding items in the target patient information. Further, rehabilitation histories of the past patients are associated with the past information.

The ranking unit 3 extracts at least one piece of past information from a plurality of pieces of past information based on the degree of similarity calculated by the similarity calculation unit 2, and ranks the at least one piece of past information according to a predetermined ranking rule. Specifically, the ranking unit 3 extracts pieces of past information for which the degrees of similarity are higher than a predetermined criterion, and ranks the group of extracted pieces of past information.

This ranking rule is, for example, a ranking made by sorting based on the degrees of improvement in the ability values of the past patients after performing rehabilitation, but it is not limited to this ranking. That is, the ranking may be any type of ranking made by sorting based on arbitrary information included in the past information. For example, the sorting may be, for example, sorting based on the numbers of years of experience of therapists who performed rehabilitation therapies for the past patients.

The determination unit 4 determines, as a rehabilitation plan for the target patient, a rehabilitation history of a past patient associated with the past information according to the result of the ranking of the pieces of past information made by the ranking unit 3. For example, the determination unit 4 determines, as the rehabilitation plan for the target patient, a rehabilitation history of a past patient associated with past information of which the rank is in a predetermined range (e.g., top k ranks (k is an integer equal to or greater than one)). Note that the determination unit 4 may perform control so as to output the rehabilitation history, and may further perform control so as to output a part or all of the past information associated with the rehabilitation history together with the rehabilitation history.

As described above, according to the rehabilitation planning apparatus 1, pieces of past information of past patients having features similar to those of the target patient are ranked. Then, the rehabilitation history associated with the past information selected according to the result of the ranking is determined to be the rehabilitation plan for the target patient. That is, it is possible to rank rehabilitation histories of past patients having features similar to those of the target patient according to the desired viewpoint, and then to determine one of the ranked rehabilitation histories as a rehabilitation plan for the target patient. Therefore, a therapist can create a rehabilitation plan for the target patient by referring to the determined rehabilitation plan. Alternatively, the therapist can determine the determined rehabilitation plan itself as the rehabilitation plan for the target patient. As described above, according to the rehabilitation planning apparatus 1, it is possible to provide useful information for examining a rehabilitation plan, and thereby to efficiently create the rehabilitation plan.

### First Example Embodiment

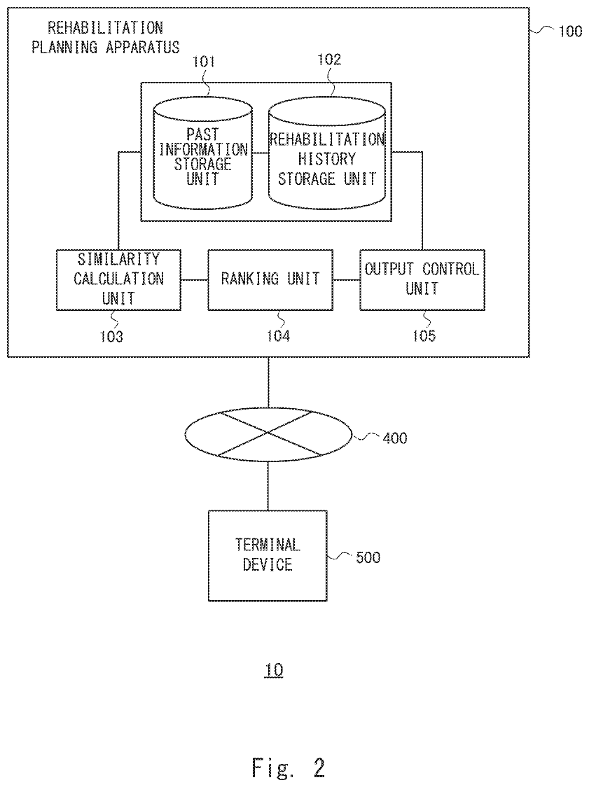

An example embodiment according to the present invention will be described hereinafter with reference to the drawings. FIG. 2 is a block diagram showing an example of a configuration of a rehabilitation planning system 10 according to a first example embodiment. The rehabilitation planning system 10 includes a rehabilitation planning apparatus 100 and a terminal device 500, and they are connected to each other through a network 400 wirelessly or through a cable so that they can communicate with each other.

The rehabilitation planning apparatus 100 is configured, for example, as a server. Further, the terminal device 500 is an arbitrary terminal such as a personal computer, a tablet-type terminal, or a smartphone. The terminal device 500 is equipped with an input device and an output device, and hence is able to receive information to be transmitted to the rehabilitation planning apparatus 100 and output (display) information received from the rehabilitation planning apparatus 100.

Note that although only one terminal device 500 is shown in FIG. 2, the rehabilitation planning system 10 may include a plurality of terminal devices 500.

As shown in FIG. 2, the rehabilitation planning apparatus 100 includes a past information storage unit 101, a rehabilitation history storage unit 102, a similarity calculation unit 103, a ranking unit 104, and an output control unit 105. The rehabilitation planning apparatus 100 is an apparatus for assisting a therapist or the like to create a rehabilitation plan for a target patient. This rehabilitation plan is information indicating a schedule of rehabilitation that the target patient, who is a patient in a certain facility (or an institution), will perform. In this example embodiment, the above-described certain facility is, for example, but not limited to, a convalescent rehabilitation hospital to which patients who are transferred from an acute-phase hospital are admitted.

In the past information storage unit 101, past information for each past patient is stored. Information about each item in the past information and information about each item in the target patient information (which will described later) are, for example, expressed by numerical codes. Note that the past information used in the processing performed in the rehabilitation planning apparatus 100 preferably does not include past information about unusual patients (e.g., patients having special circumstances) because such information is likely to be useless for rehabilitation of a target patient.

In the rehabilitation history storage unit 102, a rehabilitation history of each past patient is stored. For each past patient, the past information stored in the past information storage unit 101 and the rehabilitation history stored in the rehabilitation history storage unit 102 are associated with each other.

Note that although the past information storage unit 101 and the rehabilitation history storage unit 102 are shown as separate components in the example shown in FIG. 2, they may be implemented (i.e., constructed) as a one storage unit. Further, although the rehabilitation planning apparatus 100 includes the past information storage unit 101 and the rehabilitation history storage unit 102 in this example embodiment, the past information storage unit 101 and the rehabilitation history storage unit 102 may be implemented by an external apparatus(es). In such a case, the rehabilitation planning apparatus 100 may acquire the past information and the rehabilitation history from this external apparatus.

In this example embodiment, the past information includes patient's attributes, the name of a disease, symptoms, ability values, and an individual target. However, these information items are merely examples, and the past information is not limited to them. Specifically, the patient's attributes include, for example, any attribute information such as the age, the gender, and social information of the patient. Note that the social information is information representing the social states of the patient, and includes a family structure, the presence/absence of a roommate(s), the place of residence, the type of the building of the home (e.g., whether the building is a condominium or a two-story detached house), information about patient's medical insurance, and information about patient's nursing-care insurance.

The ability values are ability values related to patient's activities in daily life, and for example, ability values related to ADL (Activities of Daily Living) or IADL (Instrumental Activities of Daily Living). In this example embodiment, the ability values included in the past information are, specifically, evaluation scores in respective evaluation items included in an FIM (Function Independence Measure). However, other types of ability values may also be used.

In this example embodiment, the ability values included in the past information are information (e.g., time-series data) indicating temporal changes in the evaluation values in respective evaluation items in the FIM. That is, the past information includes histories of ability values. In this example embodiment, the histories of ability values include histories of ability values in rehabilitation (convalescent rehabilitation) performed in a convalescent rehabilitation hospital. They include ability values of the past patient before performing rehabilitation indicated in a rehabilitation history associated with the past information (a rehabilitation history stored in the rehabilitation history storage unit 102) and ability values after performing the rehabilitation. Further, in this example embodiment, the past information includes, as the histories of ability values, not only histories of ability values in convalescent rehabilitation, but also histories of ability values in rehabilitation performed in an acute-phase hospital (i.e., acute-phase rehabilitation). Although the ability values are respective values for a plurality of types of abilities (values in respective items in the FIM) in this example embodiment, they can also be ability values for one type of ability.

The individual target is information indicating an individual target of a patient in rehabilitation. For example, the individual target may be, but is not limited to, any of the below-shown items.
“Be able to cross a street within a time during which a traffic light is green”
“Be able to walk at a quick pace”
“Be able to be reinstated as a clerical worker”
“Be able to live alone without nursing care”
“A score in each classification item in ADL-related indices such as the FIM becomes better than a predetermined value”
“A level of required support or a level of required care in the nursing-care field becomes better than a predetermined value”
“Be able to be reinstated as a clerical worker who mainly operates a desk-top calculator”
“No assistance is required, except for bathing, and be able to live alone in his/her house as long as he/she receives a certain nursing-care service”
“Be able to walk while avoiding obstacles without feeling of wrongness as being observed by people around him/her”
“Be able to go up and down stairs while holding a light object in his/her house”.

In this example embodiment, the rehabilitation history storage unit 102 specifically stores, as a rehabilitation history, a rehabilitation history in a convalescent rehabilitation hospital. The rehabilitation history is, for example, time-series data about contents of rehabilitation at predetermined intervals (e.g., on a weekly basis). The contents of rehabilitation include, for example, tasks that the patient desires to accomplish through the rehabilitation and contents (programs) of practices for accomplishing the tasks. Regarding the tasks, a superordinate task(s) and a subordinate task(s) may be set. Further, the rehabilitation history may also include a time when an outing event occurred. Note that the outing event means a certain event that involves an outing, such as an overnight stay in a place other than his/her hospital or a temporary stay in his/her house in which he/she performs rehabilitation. Further, the rehabilitation history may also include various information items such as identification information of the therapist who performed the rehabilitation therapy and a place where the rehabilitation is performed.

The similarity calculation unit 103 corresponds to the similarity calculation unit 2 shown in FIG. 1, and calculates the degree of similarity between each of a plurality of pieces of past information of a respective one of a plurality of past patients stored in the past information storage unit 101 and the target patient information. Note that, for example, the target patient information is acquired as described below.

For example, the therapist inputs target patient information to the terminal device 500, and the terminal device 500 transmits the input target patient information to the rehabilitation planning apparatus 100. The target patient information received by the rehabilitation planning apparatus 100 is input to the similarity calculation unit 103.

In this example embodiment, as the target patient information, the same type of information as that of the past information is input to the similarity calculation unit 103. That is, in this example embodiment, similarly to the past information, the target patient information includes patient's attributes, the name of a disease, symptoms, ability values, an individual target, and the like. However, these information items are merely examples, and the target patient information is not limited to them. Note that the ability values included in the target patient information are, for example, information (e.g., time-series data) indicating temporal changes in the evaluation values in respective evaluation items in the FIM. As described above, the target patient information includes histories of ability values. In this example embodiment, the histories of ability values included in the target patient information includes at least histories of ability values in rehabilitation (acute-phase rehabilitation) performed in an acute-phase rehabilitation hospital. However, when the target patient has already performed rehabilitation (convalescent rehabilitation) in a convalescent rehabilitation hospital, the histories of ability values may further include histories of ability values in the rehabilitation (convalescent rehabilitation) in the convalescent rehabilitation hospital.

The similarity calculation unit 103 may calculate the degree of similarity between the target patient information and the past information by calculating a distance between a vector corresponding to the target patient information and a vector corresponding to the past information. In such a case, it means that the smaller the distance is, the higher the degree of similarity is. Note that the similarity calculation unit 103 may calculate a distance while assigning a predetermined weight to each element of the vector (i.e., to each item in the past information and the target patient information). However, the above-described calculation method is merely an example, and other arbitrary calculation methods may be used to determine similarity between features of the target patient and those of the past patients. Note that when the input target patient information includes only some of the items included in the past information, the similarity calculation unit 2 may compare only these items in the target patient information with those in the past information.

Note that, in particular, in this example embodiment, each of the past information and the target patient information includes histories of ability values for a predetermined rehabilitation period (specifically, for an acute phase rehabilitation period), and the similarity calculation unit 103 calculates the degrees of similarity of at least the histories of ability values for the predetermined rehabilitation period. More specifically, the similarity calculation unit 103 calculates the degree of similarity of a changing pattern of an ability value for the predetermined rehabilitation period. There is a tendency that patients who have histories (changing patterns) of ability values similar to each other will recover in manners similar to each other when they perform the same contents of rehabilitation as each other. Therefore, by taking the degree of similarity between histories (changing patterns) of ability values for rehabilitation into consideration, it is possible to determine the degree of similarity between the past patient and the target patient more accurately.

Further, in this example embodiment, each of the past information and the target patient information includes a target (an individual target) in rehabilitation, and the similarity calculation unit 103 calculates at least the degree of similarity between the targets. There is a tendency that patients who have targets in rehabilitation similar to each other will recover in manners similar to each other when they perform the same contents of rehabilitation as each other. Therefore, by taking the degree of similarity between targets in rehabilitation into consideration, it is possible to determine the degree of similarity between the past patient and the target patient more accurately.

Note that although the similarity calculation unit 103 determines the degree of similarity between histories of ability values for a predetermined rehabilitation period and the degree of similarity between individual targets in this example embodiment as described above, the similarity calculation unit 103 may determine only one of them, or may determine neither of them. That is, it is sufficient if the similarity calculation unit 103 calculates a degree of similarity based on any of the items included in each of the past information and the target patient information.

The ranking unit 104 corresponds to the ranking unit 3 shown in FIG. 1. Therefore, the ranking unit 104 extracts pieces of past information from a plurality of pieces of past information based on the degree of similarity calculated by the similarity calculation unit 103, and ranks the extracted pieces of past information according to a predetermined ranking rule. In this example embodiment, the ranking unit 104 extracts n pieces of past information (n is an integer equal to or greater than one) for which the degrees of similarity are higher than a predetermined criterion, and ranks the group of extracted pieces of past information. In this example embodiment, the aforementioned predetermined ranking rule is a ranking by sorting based on the degrees of improvement in the ability values of the past patients after performing rehabilitation indicated in the rehabilitation history stored in the rehabilitation history storage unit 102. That is, the ranking unit 104 ranks the pieces of past information by sorting them according to how much the ability values of the past patients similar to the target patient have recovered by performing the rehabilitation indicated in the rehabilitation history in the convalescent rehabilitation hospital.

The output control unit 105 includes the function of the determination unit 4 shown in FIG. 1, and performs control so as to output, as a rehabilitation plan for the target patient, a rehabilitation history of a past patient associated with the past information according to the result of the ranking of the pieces of past information made by the ranking unit 3. Specifically, the output control unit 105 performs control so as to output (i.e., to display) it on the display of the terminal device 500.

For example, the output control unit 105 performs control so as to output, as the rehabilitation plan for the target patient, a predetermined number of rehabilitation histories of respective past patients associated with respective pieces of past information in descending order of the aforementioned degrees of improvement in the ability values. That is, the output control unit 105 recommends, as the rehabilitation plan for the target patient, the contents of rehabilitation of the past patients by which the degrees of improvement in ability values are high. As a result, the therapist can recognize (e.g., find) rehabilitation desirable for the target patient. Note that the output control unit 105 may perform control so as to output, as examples of failures, a predetermined number of rehabilitation histories of respective past patients associated with respective pieces of past information in ascending order of the degrees of improvement in the ability values. As a result, the therapist can recognize (e.g., find) rehabilitation that is not desirable for the target patient.

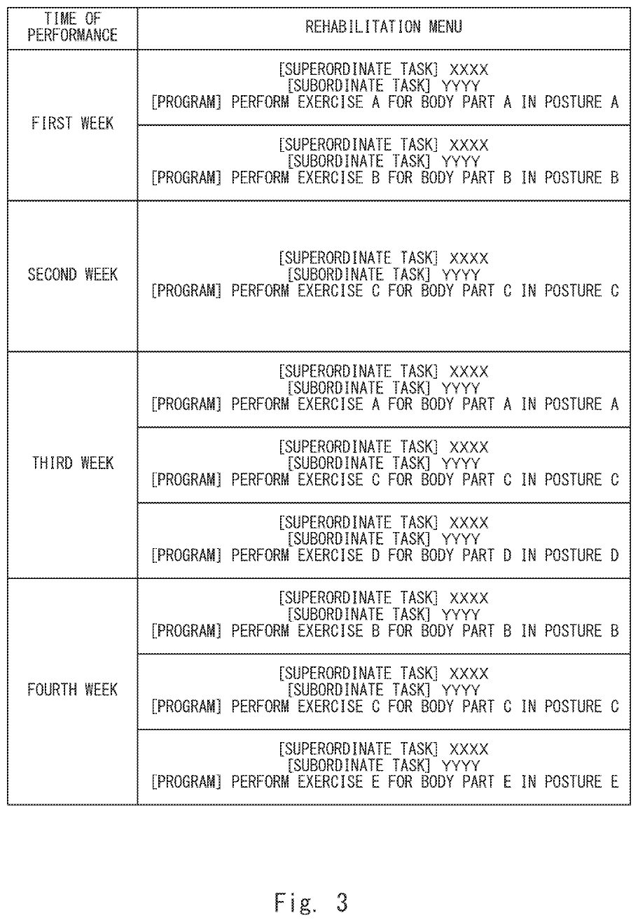

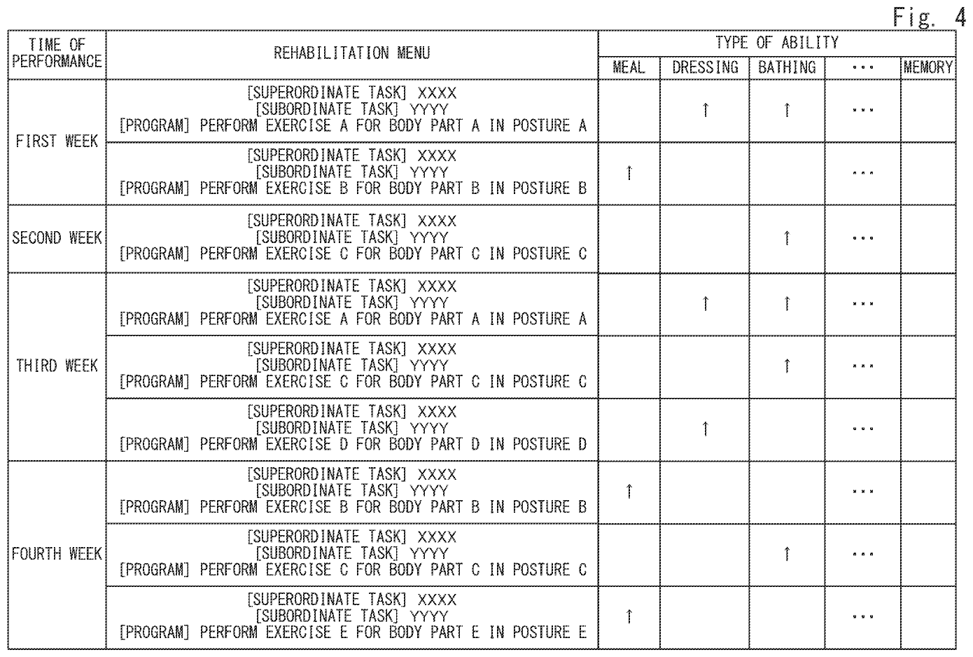

FIG. 3 is a table showing an example of a rehabilitation plan displayed on the terminal device 500 under the control of the output control unit 105. As described above, the output control unit 105 refers to (i.e., retrieves) a rehabilitation history stored in association with the past information (in the example shown in FIG. 3, a history from the first week to the fourth week after admission to a convalescent rehabilitation hospital), and displays it as a rehabilitation plan. Note that, as shown in FIG. 4, the output control unit 105 may perform control so as to output, along with the rehabilitation history of the past patient, information specifying the type of ability of which the ability value has been improved by performing rehabilitation indicated in this rehabilitation history. As described above, the past information includes, for each of a plurality of types of abilities, an ability value of the past patient before performing the rehabilitation indicated in the rehabilitation history and an ability value thereof after performing the rehabilitation. Therefore, the output control unit 105 compares, for each of a plurality of types of abilities, the ability value of the past patient before performing the rehabilitation indicated in the rehabilitation history and the ability value thereof after performing the rehabilitation, and specifies a type(s) of ability of which the ability value has improved. Then, based on the result of the specification, the output control unit 105 clearly indicates which abilities will improve. In the example shown in FIG. 4, it is shown that abilities to which upward arrows are added are those of which the ability values will improve. By clearly indicating which abilities will improve as described above, it is possible to clearly indicate the reason for presenting the rehabilitation plan. Therefore, the therapist can determine the rehabilitation plan while knowing he/she can expect which abilities will improve.

Further, for example, when a rehabilitation history of a past patient includes a time when an outing event by the past patient occurred, the output control unit 105 may perform control so as to also output the time of the occurrence of the outing event. For example, the output control unit 105 outputs information indicating the number of days (or the number of weeks) after the admission to the hospital on which the past patient stayed overnight in a place other than the hospital. In this way, the therapist can refer to the time when the outing event occurred.

Further, the output control unit may also perform control so as to output, as the rehabilitation plan for the target patient, among the rehabilitation histories of the past patients associated with the past information, those of which the times of the occurrences of outing events satisfy a predetermined condition. For example, in the case where a time when the target patient has an outing event has already been determined, the output control unit may perform control so as to output information about past patients who had outing events at similar timings. In this way, it is possible to assist the therapist or the like to create a rehabilitation plan in which the already-determined time of the occurrence of an outing event is taken into consideration.

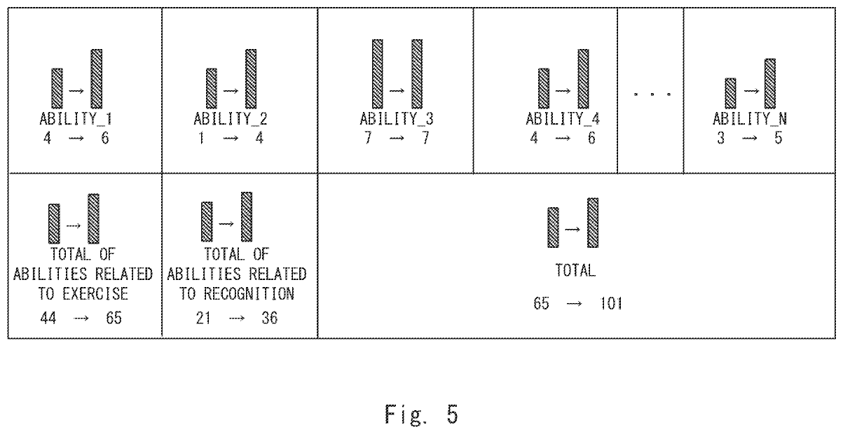

Further, the output control unit 105 may also perform control so as to output a part or all of the past information associated with the rehabilitation history along with the rehabilitation history. For example, as shown in FIG. 5, the output control unit 105 may perform control so as to display histories of ability values. Note that, in the example shown in FIG. 5, an example in which ability values of a past patient at the time of the admission to the hospital (before performing convalescent rehabilitation) and those at the time of discharge therefrom (after performing the convalescent rehabilitation) are compared with each other is shown. More specifically, in the example shown in FIG. 5, ability values are compared for each of N types of abilities. Further, in the example shown in FIG. 5, the sum totals of ability values for certain abilities are compared with each other, and the sum totals of ability values for all the abilities are also compared with each other. Note that the N types of abilities are, for example, abilities defined in evaluation items in the FIM. Examples of the abilities include how well the patient can independently perform eating movements, and how well the patient can independently change his/her upper-body clothes.

Further, the output control unit 105 may also perform control so as to output other information along with the rehabilitation history. For example, when an incident occurred for a past patient, the past information for that past patient may be associated with incident information indicating the incident that occurred. In this case, the output control unit 105 may, for example, perform control so as to further output incident information associated with at least one piece of past information extracted by the ranking unit 104. In this way, the therapist can recognize incidents that occurred in the past in patients similar to the target patient.

Further, information indicating which features of the target patient are similar to those of the past patient of which the past information has been determined to be output, or indicating which features of the target patient are dissimilar to those of the past patient may be output. That is, the output control unit 105 may perform control so as to output, among the items of information included in the past information associated with the rehabilitation history to be output as the rehabilitation plan for the target patient and those included in the target patient information, information indicating the below-shown items. For example, the output control unit 105 may perform control so as to output, in order to show similar features, information indicating items in which the degrees of similarity are larger than a predetermined first criterion. Further, the output control unit 105 may perform control so as to output, in order to show dissimilar features, information indicating items in which the degrees of similarity are smaller than a predetermined second criterion. In this way, it is possible to provide, to the therapist, information by which he/she can determine whether or not the plan proposed by the rehabilitation planning apparatus 100 is trustworthy.

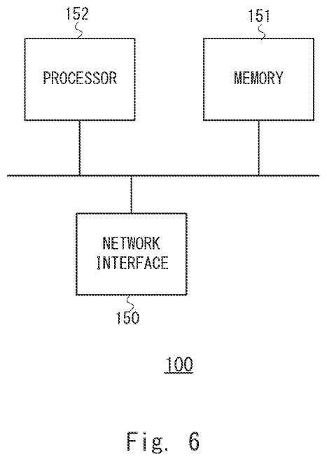

FIG. 6 is a schematic diagram showing an example of a hardware configuration of the rehabilitation planning apparatus 100. As shown in FIG. 6, the rehabilitation planning apparatus 100 includes a network interface 150, a memory 151, and a processor 152.

The network interface 150 is used to communicate with other arbitrary apparatus such as the terminal device 500. The memory 151 is composed of, for example, a combination of a volatile memory and a non-volatile memory. The memory 151 is used to store software (a computer program) including one or more instructions executed by the processor 152, and data used for various processes performed by the rehabilitation planning apparatus 100. The past information storage unit 101 and the rehabilitation history storage unit 102 shown in FIG. 2 are implemented, for example, by the memory 151, but may instead be implemented by other storage devices.

The processor 152 performs a process performed by each of the similarity calculation unit 103, the ranking unit 104, and the output control unit 105 shown in FIG. 2 by loading the software (the computer program) from the memory 151 and executing the loaded software. The processor 152 may be, for example, a microprocessor, an MPU (Micro Processor Unit), or a CPU (Central Processing Unit). The processor 152 may include a plurality of processors.

As described above, the rehabilitation planning apparatus 100 has functions as a computer. Note that, similarly, the terminal device 500 has a hardware configuration like the one shown in FIG. 6. That is, the processes performed by the terminal device 500 are implemented, for example, by having the processor execute the program.

Further, the program may be stored in various types of non-transitory computer readable media and thereby supplied to computers. The non-transitory computer readable media includes various types of tangible storage media. Examples of the non-transitory computer readable media include a magnetic recording medium (such as a flexible disk, a magnetic tape, and a hard disk drive), a magneto-optic recording medium (such as a magneto-optic disk), a CD-ROM (Read Only Memory), CD-R, CD-R/W, and a semiconductor memory (such as a mask ROM, a PROM (Programmable ROM), an EPROM (Erasable PROM), a flash ROM, and a RAM (Random Access Memory)). Further, the programs may be supplied to computers by using various types of transitory computer readable media. Examples of the transitory computer readable media include an electrical signal, an optical signal, and an electromagnetic wave. The transitory computer readable media can be used to supply programs to a computer through a wired communication line (e.g., electric wires and optical fibers) or a wireless communication line.

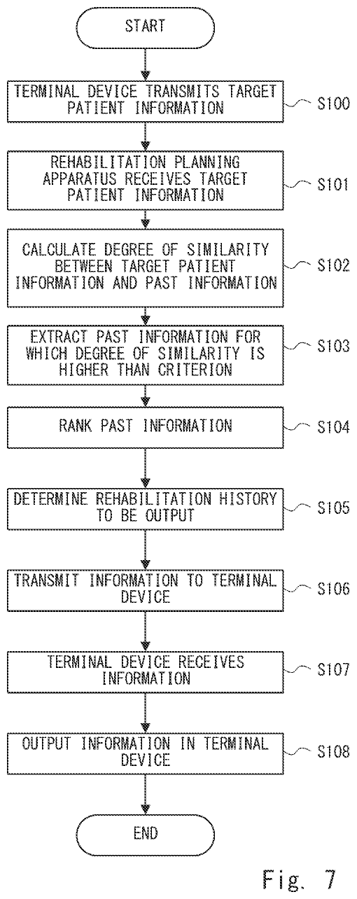

Next, a flow of operations performed by the rehabilitation planning system 10 will be described. FIG. 7 is a flowchart showing an example of the operations performed by the rehabilitation planning system 10. The flow of operations will be described hereinafter with reference to FIG. 7.

In a step S100, the terminal device 500 transmits target patient information to the rehabilitation planning apparatus 100.

Next, in a step S101, the rehabilitation planning apparatus 100 receives the target patient information. As a result, the similarity calculation unit 103 of the rehabilitation planning apparatus 100 acquires the target patient information.

Next, in a step S102, the similarity calculation unit 103 calculates the degree of similarity between the target patient information and each of pieces of past information stored in the past information storage unit 101.

Next, in a step S103, the ranking unit 104 extracts n pieces of past information for which the degrees of similarity calculated in the step S102 are higher than a predetermined criterion.

Next, in a step S104, the ranking unit 104 ranks the pieces of past information extracted in the step S103.

Next, in a step S105, the output control unit 105 determines rehabilitation histories to be output. The output control unit 105 selects pieces of past information according to the result of the ranking made in the step S104, and determines rehabilitation histories of past patients associated with the selected pieces of past information as those to be output.

Next, in a step S106, the output control unit 105 transmits the rehabilitation histories of the past patients to the terminal device 500. Note that, as described above, when information other than the rehabilitation histories is also output, the output control unit 105 also transmits that information to the terminal device 500.

Next, in a step S107, the terminal device 500 receives the information.

Then, in a step S108, the terminal device 500 outputs the received information. Specifically, the terminal device 500 displays the received information, for example, on the display thereof.

The rehabilitation planning system 10 according to the first example embodiment has been described above. According to this system, a rehabilitation plan for a target patient is provided based on past information. Therefore, it is possible to efficiently create a rehabilitation plan. In particular, in this system, it is possible, by the ranking unit 104, to rank past patients by sorting based on the degrees of improvement in their ability values after performing rehabilitation. Therefore, it is possible to present, to a therapist, a rehabilitation plan by which ability values can be improved. Therefore, for example, even a therapist with a small number of years of experience can make a rehabilitation plan by which ability values of a target patient can be improved. As a result, it is possible to reduce variations among the results (or effects) in regard to the recoveries of patients by therapists. Further, in this system, it is possible to present a rehabilitation plan by which ability value of a target patient can be improved. Therefore, it can also be expected to be effective to educate therapists who are not skilled in making appropriate rehabilitation plans by having them use this system.

### Second Example Embodiment

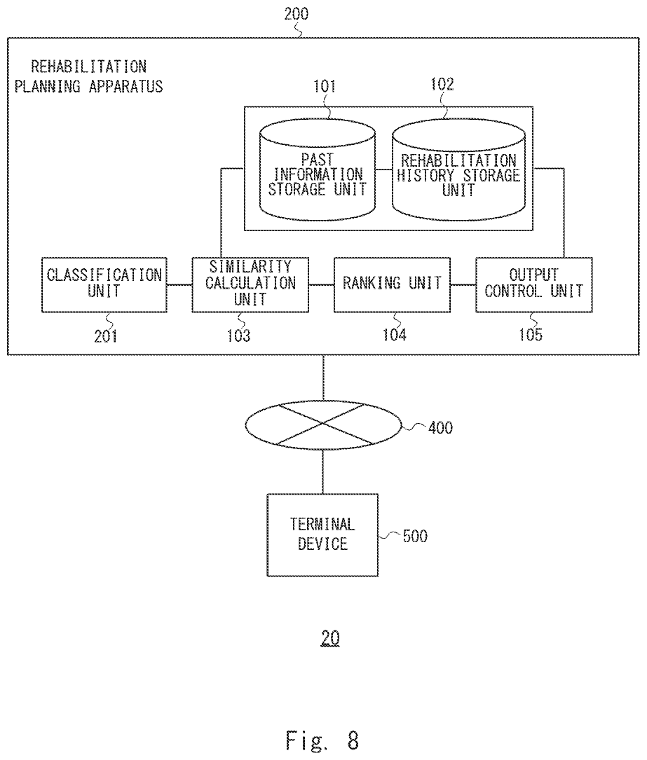

Next, a second example embodiment will be described. A rehabilitation planning system 20 according to a second example embodiment differs from the rehabilitation planning system 10 according to the first example embodiment in that the rehabilitation planning apparatus 100 is replaced by a rehabilitation planning apparatus 200. FIG. 8 is a block diagram showing an example of a configuration of the rehabilitation planning system 20 according to the second example embodiment. As shown in FIG. 8, the rehabilitation planning apparatus 200 differs from the rehabilitation planning apparatus 100 in the first example embodiment in that a classification unit 201 is added. Features that differ from those in the first example embodiment will be described hereinafter, while redundant descriptions will be omitted as appropriate. The process performed by the classification unit 201 is also processed, for example, by having the processor 152 load the software (the computer program) from the memory 151 and execute the loaded software.

The classification unit 201 classifies pieces of past information and target patient information based on information included in the pieces of past information and in the target patient information, and excludes, from the information to be output, information related to pieces of past information that have been classified into categories different from the category in which the target patient information has been classified. In the first example embodiment, the degree of similarity between the target patient information and each of all the pieces of past information stored in the past information storage unit 101 is calculated. However, in this case, there may be cases where even when a past patient has past information that is calculated (i.e., determined) to have a high degree of similarity to the target patient information of the target patient, his/her actual characteristics for rehabilitation are not similar to those of the target patient. In regard to this matter, the inventors have found that it is possible to exclude past patients of which characteristics for rehabilitation are not similar to those of the target patient by classifying pieces of past information and target patient information while focusing on information (items) included in the pieces of past information and the target patient information. Therefore, in this example embodiment, when pieces of past information and target patient information have been classified while focusing on information included in the pieces of past information and in the target patient information, only the information related to past patients of which pieces of past information are classified in the same category as that of the target patient information is determined to be information to be output. Note that, for example, histories of ability values may be used as information used for the classification. More specifically, the pieces of past information and the target patient information may be classified based on changing patterns of ability value during a predetermined rehabilitation period (e.g., a period during which acute-phase rehabilitation was performed). Further, targets (individual targets) for rehabilitation may be used as the information used for the classification. However, they are merely examples, and the pieces of past information and the target patient information may be classified based on other information included in the past information and in the target patient information.

The classification unit 201 performs a clustering process for the pieces of past information and the target patient information while focusing on the aforementioned information, and thereby classifies each of the pieces of past information and the target patient information into one of categories. The classification unit 201 performs a filtering process so that information about past patients having pieces of past information that have been classified in the same category as that of the target patient information are determined to be information to be output.

For example, as shown in FIG. 8, in the case where the process by the classification unit 201 is performed as a process that is performed before the process by the similarity calculation unit 103, the classification unit 201 notifies the similarity calculation unit 103 of the pieces of past information classified into the same category as that of the target patient information. The similarity calculation unit 103 calculates the degree of similarity between each of the pieces of past information classified in the same category as that of the target patient information and the target patient information. That is, in this case, the degree of similarity is not calculated for any of the pieces of past information classified in categories different from that of the target patient information, and the pieces of past information and rehabilitation histories associated with them are not used for the processes performed by the ranking unit 104 and the output control unit 105. Note that since the classification unit 201 is a component that provides a function as a filter for filtering information to be output, its process does not necessarily have to be performed before the process performed by the similarity calculation unit 103. For example, the process performed by the classification unit 201 may be performed after the process performed by the similarity calculation unit 103 and before the process performed by the ranking unit 104, or may be performed after the process performed by the ranking unit 104. Therefore, although this example embodiment is described on the assumption that the process performed by the classification unit 201 is performed before the process performed by the similarity calculation unit 103, the order of processes is not limited to this example.

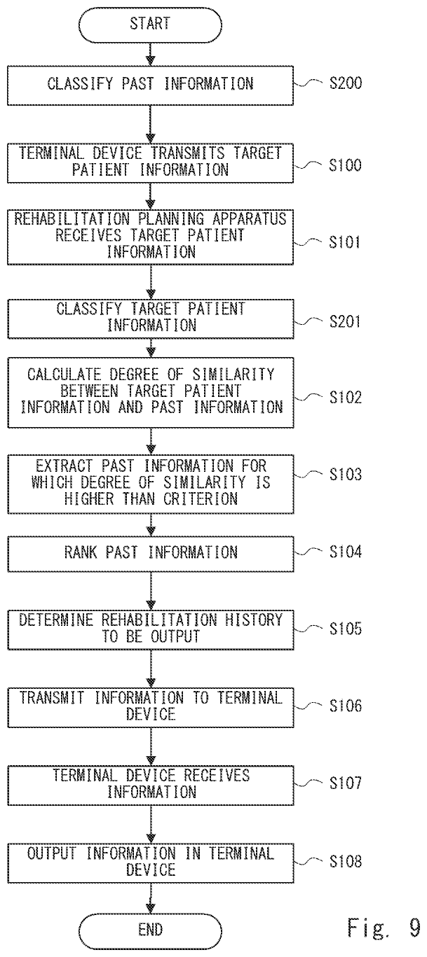

FIG. 9 is a flowchart showing an example of operations performed by the rehabilitation planning system 20. The flowchart shown in FIG. 9 differs from that shown in FIG. 7 in that steps S200 and S201 representing processes performed by the classification unit 201 are added. Features that differ from those shown in FIG. 7 will be described hereinafter, while descriptions of the same features as those shown in FIG. 7 will be omitted as appropriate.

In a step S200, the classification unit 201 performs a clustering process for pieces of past information stored in the past information storage unit 101, and thereby classifies these pieces of past information.

Next, in a step S100, the terminal device 500 transmits target patient information to the rehabilitation planning apparatus 100, and in a step S101, the rehabilitation planning apparatus 100 receives the target patient information.

Next, in a step S201, the classification unit 201 performs a clustering process for the target patient information received in the step S101, and thereby classifies the target patient information. Then, the classification unit 201 notifies the similarity calculation unit 103 of the pieces of past information that have been classified into the same category as that of the target patient information.

Next, in a step S102, the similarity calculation unit 103 calculates the degree of similarity between the target patient information and each of pieces of past information. However, the similarity calculation unit 103 processes, among the pieces of past information stored in the past information storage unit 101, only those that have been classified in the same category as that of the target patient information. After the step S102, the processes in a step S103 and the subsequent steps are performed in a manner similar to that shown in FIG. 7.

The second example embodiment has been described above. In this example embodiment, because of the process performed by the classification unit 201, a filtering process is performed so that, among various pieces of past information, only the pieces of past information that have been classified in the same category as that of the target patient information are referred to (i.e., are used). Therefore, it is possible to prevent information about past patients of which characteristics for rehabilitation are not similar to those of the target patient from being referred to (i.e., being used). Accordingly, it is possible to output more appropriate information as compared to the case where the classification unit 201 is not provided.

### Third Example Embodiment

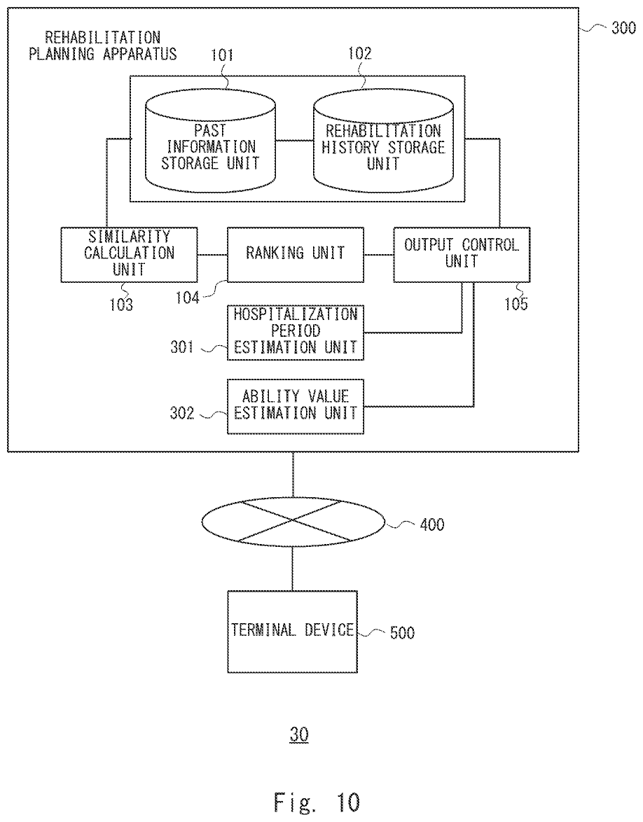

Next, a third example embodiment will be described. A rehabilitation planning system 30 according to a third example embodiment differs from the rehabilitation planning system 10 according to the first example embodiment in that the rehabilitation planning apparatus 100 is replaced by a rehabilitation planning apparatus 300. FIG. 10 is a block diagram showing an example of a configuration of the rehabilitation planning system 30 according to the third example embodiment. As shown in FIG. 10, the rehabilitation planning apparatus 300 differs from the rehabilitation planning apparatus 100 according to the first example embodiment in that a hospitalization period estimation unit 301 and an ability value estimation unit 302 are added. The process performed by each of the hospitalization period estimation unit 301 and the ability value estimation unit 302 is also processed, for example, by having the processor 152 load the software (the computer program) from the memory 151 and execute the loaded software. Features that differ from those in the first example embodiment will be described hereinafter, while redundant descriptions will be omitted as appropriate.

The hospitalization period estimation unit 301 estimates a hospitalization period in a predetermined facility (or a predetermined institution) (specifically, for example, in a convalescent rehabilitation hospital) on the assumption that the target patient performs rehabilitation indicated in the rehabilitation history of the past patient of which the past information has been determined to be output. The hospitalization period estimation unit 301, for example, estimates the hospitalization period in the below-shown manner. Note that the following description will be given under the assumption that the past information of a past patient includes his/her hospitalization period in a predetermined facility (specifically, for example, in a convalescent rehabilitation hospital).

For example, the hospitalization period estimation unit 301 may estimate (i.e., determine) a hospitalization period included in the past information as a hospitalization period that is expected under the assumption that the target patient performs the rehabilitation indicated in the rehabilitation history associated with this past information. That is, the hospitalization period estimation unit 301 may cite the hospitalization period indicated in the past information.

Further, for example, the hospitalization period estimation unit 301 may estimate the hospitalization period by using a prediction model that has undergone a learning process in advance. Specifically, for example, the hospitalization period estimation unit 301 may estimate the hospitalization period by using a prediction model that has undergone a learning process in advance by using the past information and the rehabilitation history associated with the past information. In such a case, the prediction model has undergone a learning process in advance by using, as training data, hospitalization periods of past patients included in the past information, information about other features of the past patients included in the past information, and rehabilitation histories of the past patients associated with the past information. Note that the information about the other features is any other information included in the past information other than the hospitalization period of the past patient, such as patient's attributes, the name of a disease, symptoms, ability values, and an individual target. For example, some or all of these information items may be used as information about other features for the learning process of the model. Data provided to the model has already been converted into numerically codes. The predictive model is a model that, when information about other features and a rehabilitation history are input, outputs a hospitalization period. For example, the model is a support vector machine (SVM: Support vector machine) or support vector regression (SVR: Support Vector Regression). However, the model is not limited to these examples, and may be other machine learning models such as a neural network.

When the hospitalization period estimation unit 301 estimates the hospitalization period by using the above-described prediction model, the hospitalization period estimation unit 301 inputs, to the prediction model, information about other features included in the target patient information and the rehabilitation history of the past patient of which the past information is to be output. Then, the hospitalization period estimation unit 301 determines the hospitalization period output by the prediction model as the hospitalization period that is expected under the assumption that the target patient performs the rehabilitation indicated in the rehabilitation history of the past patient of which the past information is to be output.

The ability value estimation unit 302 estimates an ability value that is expected under the assumption that the target patient performs the rehabilitation indicated in the rehabilitation history of the past patient of which the past information is to be output. For example, the ability value estimation unit 302 estimates the ability value in the below-shown manner. Note that the following description will be given on the assumption that the past information of the past patient includes the ability value of the past patient after performing the rehabilitation indicated in the rehabilitation history associated with the past information.

For example, the ability value estimation unit 302 may estimate (i.e., determine) an ability value included in the past information as an ability value that is expected under the assumption that the target patient performs the rehabilitation indicated in the rehabilitation history associated with this past information. That is, the ability value estimation unit 302 may cite the ability value indicated in the past information.

Further, for example, the ability value estimation unit 302 may estimate the ability value by using a prediction model that has undergone a learning process in advance. Specifically, for example, the ability value estimation unit 302 may estimate the ability value by using a prediction model that has undergone a learning process in advance by using the past information and the rehabilitation history associated with the past information. In such a case, the prediction model has undergone a learning process in advance by using, as training data, ability values after performing rehabilitation included in the past information, information about other features of the past patients included in the past information, and rehabilitation histories of the past patients associated with the past information. Note that the ability value after performing rehabilitation is an ability value after performing rehabilitation indicated in the rehabilitation history used for the learning. The information about the other features is any other information included in the past information other than the ability value of the past patient after performing the rehabilitation. For example, some or all of patient's attributes, the name of a disease, symptoms, ability values, an individual target, and the like are used as information about other features for the learning process of the model. Data provided to the model has already been converted into numerically codes. This predictive model is a model that, when information about other features and a rehabilitation history are input, outputs an ability value. Specifically, the model outputs, for example, an evaluation value for each of evaluation items in the FIM. For example, the model is a support vector machine or support vector regression. However, the model is not limited to these examples, and may be other machine learning models such as a neural network.

When the ability value estimation unit 302 estimates an ability value by using the above-described prediction model, the ability value estimation unit 302 inputs, to the prediction model, information about other features included in the target patient information and the rehabilitation history of the past patient of which the past information is to be output. Then, the ability value estimation unit 302 determines the ability value output by the prediction model as the ability value that is expected under the assumption that the target patient performs the rehabilitation indicated in the rehabilitation history of the past patient of which the past information is to be output.

Note that when the past information of the past patient includes an ability value after the discharge from the predetermined facility (specifically, for example, from a convalescent rehabilitation hospital), the ability value estimation unit 302 may estimate the ability value after the discharge.

The output control unit 105 performs control so as to output the estimated hospitalization period and the ability value along with the rehabilitation history of the past patient.

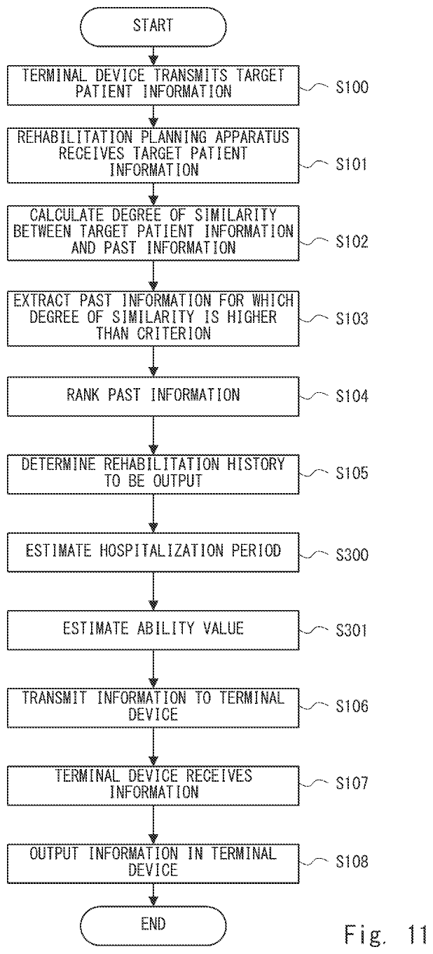

FIG. 11 is a flowchart showing an example of operations performed by the rehabilitation planning system 30. The flowchart in FIG. 11 differs from that shown in FIG. 7 in that a step S300 representing the process performed by the hospitalization period estimation unit 301 and a step S301 representing the process performed by the ability value estimation unit 302 are added. Features that differ from those shown in FIG. 7 will be described hereinafter, while descriptions of the same features as those shown in FIG. 7 will be omitted as appropriate.

In this example embodiment, the process proceeds to the step S300 after the step S105.

In the step S300, the hospitalization period estimation unit 301 estimates a hospitalization period that is expected under the assumption that the target patient performs the rehabilitation indicated in the rehabilitation history of the past patient of which the past information has been determined to be output in the step S105. The result of the estimation by the hospitalization period estimation unit 301 will be transmitted, as the information to be output, to the terminal device 500.

In the step S301, the ability value estimation unit 302 estimates an ability value that is expected under the assumption that the target patient performs the rehabilitation indicated in the rehabilitation history of the past patient of which the past information has been determined to be output in the step S105. The result of the estimation by the ability value estimation unit 302 will be transmitted, as the information to be output, to the terminal device 500.

The process proceeds to the step S106 after the process in the step S301. That is, after the steps S300 and S301, the processes in a step S106 and the subsequent steps are performed in a manner similar to that shown in FIG. 7.

The third example embodiment has been described above. In this example embodiment, the results of the estimations of a hospitalization period and an ability value under the assumption that the target patient performs rehabilitation are output. Therefore, it is possible to provide more useful information when creating a rehabilitation plan. Note that although a configuration in which both the hospitalization period and the ability value are estimated is shown in this example embodiment, only one of them may be estimated. Further, the estimation of the hospitalization period or the ability value may be performed in the second example embodiment. That is, it is also possible to implement an example embodiment in which the second and third example embodiments are combined with each other.

Note that the present invention is not limited to the above-described example embodiments and various modifications can be made within the scope and spirit of the invention.

Further, the whole or part of the example embodiments disclosed above can be described as, but not limited to, the following supplementary notes.

### (Supplementary Note 1)

A rehabilitation planning apparatus comprising:

similarity calculation means for calculating a degree of similarity between each of a plurality of pieces of past information and target patient information, each of the plurality of pieces of past information being information about a respective one of a plurality of past patients who performed rehabilitation in a past, and the target patient information being information about a target patient;

ranking means for extracting at least one piece of past information from the plurality of pieces of past information based on the degree of similarity, and ranking the at least one piece of past information according to a predetermined ranking rule; and

determination means for determining, as a rehabilitation plan for the target patient, a rehabilitation history of the past patient associated with the past information according to a result of the ranking of the pieces of past information.

### (Supplementary Note 2)

The rehabilitation planning apparatus described in Supplementary note 1, wherein

each of the pieces of past information and the target patient information includes a history of an ability value for a predetermined rehabilitation period, and

the similarity calculation means calculates at least a degree of similarity of the history of the ability value.

### (Supplementary Note 3)

The rehabilitation planning apparatus described in Supplementary note 1 or 2, wherein

each of the pieces of past information and the target patient information includes a target in rehabilitation, and

the similarity calculation means calculates at least a degree of similarity of the target.

### (Supplementary Note 4)

The rehabilitation planning apparatus described in any one of Supplementary notes 1 to 3, further comprising classification means for classifying the pieces of past information and the target patient information based on information included in the pieces of past information and in the target patient information, and excluding, from information to be processed, information related to pieces of past information that have been classified into a category different from a category in which the target patient information has been classified.

### (Supplementary Note 5)

The rehabilitation planning apparatus described in Supplementary note 4, wherein

each of the pieces of past information and the target patient information includes a history of an ability value for a predetermined rehabilitation period, and

the information used for the classification is the history of the ability value.

### (Supplementary Note 6)

The rehabilitation planning apparatus described in Supplementary note 4, wherein

each of the pieces of past information and the target patient information includes a target in rehabilitation, and

the information used for the classification is the target in the rehabilitation.

### (Supplementary Note 7)

The rehabilitation planning apparatus described in any one of Supplementary notes 1 to 6, wherein

the past information includes an ability value of the past patient before performing rehabilitation indicated in the rehabilitation history and an ability value thereof after performing the rehabilitation, and

the predetermined ranking rule is sorting based on a degree of improvement in the ability value of the past patient after performing the rehabilitation indicted in the rehabilitation history.

### (Supplementary Note 8)

The rehabilitation planning apparatus described in Supplementary note 7, wherein the determination means determines, as a rehabilitation plan for the target patient, a predetermined number of rehabilitation histories of respective past patients associated with respective pieces of past information in descending order of the degree of improvement.

### (Supplementary Note 9)

The rehabilitation planning apparatus described in Supplementary note 7, wherein the determination means determines, as examples of failures, predetermined number of rehabilitation histories of respective past patients associated with respective pieces of past information in ascending order of the degree of improvement.

### (Supplementary Note 10)

The rehabilitation planning apparatus described in any one of Supplementary notes 1 to 9, wherein

when an incident has occurred for the past patient, the past information is associated with incident information indicating the incident that has occurred, and

the determination means specifies the incident information associated with at least one extracted piece of past information.

### (Supplementary Note 11)

The rehabilitation planning apparatus described in any one of Supplementary notes 1 to 10, wherein

the past information includes, for each of a plurality of types of abilities, an ability value of the past patient before performing rehabilitation indicated in the rehabilitation history and an ability value thereof after performing the rehabilitation, and

the determination means specifies a type of ability of which an ability value is improved by performing the rehabilitation indicated in the rehabilitation history of the past patient that has been determined to be the rehabilitation plan for the target patient.

### (Supplementary Note 12)

The rehabilitation planning apparatus described in any one of Supplementary notes 1 to 11, further comprising hospitalization period estimation means for estimating a hospitalization period under an assumption that the target patient performs the rehabilitation indicated in the rehabilitation history.

### (Supplementary Note 13)

The rehabilitation planning apparatus described in Supplementary note 12, wherein

the past information includes a hospitalization period of the past patient, and

the hospitalization period estimation means estimates the hospitalization period included in the past information as a hospitalization period that is expected under the assumption that the target patient performs the rehabilitation indicated in the rehabilitation history associated with the past information.

### (Supplementary Note 14)

The rehabilitation planning apparatus described in Supplementary note 12, wherein the hospitalization period estimation means estimates the hospitalization period by using a prediction model that has undergone a learning process in advance.

### (Supplementary Note 15)

The rehabilitation planning apparatus described in any one of Supplementary notes 1 to 14, further comprising ability value estimation means for estimating an ability value under an assumption that the target patient has performs the rehabilitation indicated in the rehabilitation history.

### (Supplementary Note 16)

The rehabilitation planning apparatus described in Supplementary note 15, wherein

the past information includes an ability value of the past patient after performing the rehabilitation indicted in the rehabilitation history, and

the ability value estimation means estimates the ability value included in the past information as an ability value that is expected under the assumption that the target patient performs rehabilitation indicated in the rehabilitation history associated with the past information.

### (Supplementary Note 17)

The rehabilitation planning apparatus described in Supplementary note 15, wherein the ability value estimation means estimates the ability value by using a prediction model that has undergone a learning process in advance.

### (Supplementary Note 18)

The rehabilitation planning apparatus described in any one of Supplementary notes 1 to 17, wherein the rehabilitation history includes a time of an occurrence of an outing event of a patient.

### (Supplementary Note 19)

The rehabilitation planning apparatus described in Supplementary note 18, wherein the determination means determines, as the rehabilitation plan for the target patient, among the rehabilitation histories of the past patients associated with the past information, a rehabilitation history of which the time of the occurrence of the outing event satisfies a predetermined condition.

### (Supplementary Note 20)

The rehabilitation planning apparatus described in any one of Supplementary notes 1 to 19, wherein the determination means specifies, among items of information included in the past information associated with the rehabilitation history that has been determined to be the rehabilitation plan for the target patient and in the target patient information, an item for which the degree of similarity is larger than a predetermined first criterion or an item for which the degree of similarity is smaller than a predetermined second criterion.

### (Supplementary Note 21)

A rehabilitation planning system comprising a rehabilitation planning apparatus, and a terminal device, wherein

the rehabilitation planning apparatus comprises:

similarity calculation means for calculating a degree of similarity between each of a plurality of pieces of past information and target patient information, each of the plurality of pieces of past information being information about a respective one of a plurality of past patients who performed rehabilitation in a past, and the target patient information being information about a target patient input from the terminal device;

ranking means for extracting at least one piece of past information from the plurality of pieces of past information based on the degree of similarity, and ranking the at least one piece of past information according to a predetermined ranking rule; and

output control means for performing control so as to output, as a rehabilitation plan for the target patient, a rehabilitation history of the past patient associated with the past information to the terminal device according to a result of the ranking of the pieces of past information.

### (Supplementary Note 22)

The rehabilitation planning system described in Supplementary note 21, wherein

each of the pieces of past information and the target patient information includes a history of an ability value for a predetermined rehabilitation period, and

the similarity calculation means calculates at least a degree of similarity of the history of the ability value.

### (Supplementary Note 23)

The rehabilitation planning system described in Supplementary note 21 or 22, further comprising hospitalization period estimation means for estimating a hospitalization period under an assumption that the target patient performs the rehabilitation indicated in the rehabilitation history, and

the output control means performs control so as to output the estimated hospitalization period along with the rehabilitation history of the past patient.

### (Supplementary Note 24)

The rehabilitation planning system described in any one of Supplementary notes 21 to 23, further comprising ability value estimation means for estimating an ability value under an assumption that the target patient performs the rehabilitation indicated in the rehabilitation history, and

the output control means performs control so as to output the estimated ability value along with the rehabilitation history of the past patient.

### (Supplementary Note 25)

A rehabilitation planning method comprising:

calculating a degree of similarity between each of a plurality of pieces of past information and target patient information, each of the plurality of pieces of past information being information about a respective one of a plurality of past patients who performed rehabilitation in a past, and the target patient information being information about a target patient;

extracting at least one piece of past information from the plurality of pieces of past information based on the degree of similarity, and ranking the at least one piece of past information according to a predetermined ranking rule; and

determining, as a rehabilitation plan for the target patient, a rehabilitation history of the past patient associated with the past information according to a result of the ranking of the pieces of past information.

### (Supplementary Note 26)

A non-transitory computer readable medium storing a program for causing a computer to perform:

a similarity calculation step of calculating a degree of similarity between each of a plurality of pieces of past information and target patient information, each of the plurality of pieces of past information being information about a respective one of a plurality of past patients who performed rehabilitation in a past, and the target patient information being information about a target patient;

a ranking step of extracting at least one piece of past information from the plurality of pieces of past information based on the degree of similarity, and ranking the at least one piece of past information according to a predetermined ranking rule; and

a determination step of determining, as a rehabilitation plan for the target patient, a rehabilitation history of the past patient associated with the past information according to a result of the ranking of the pieces of past information.

Although the present invention is described above with reference to example embodiments, the present invention is not limited to the above-described example embodiments. Various modifications that can be understood by those skilled in the art can be made to the configuration and details of the present invention within the scope of the invention.

This application is based upon and claims the benefit of priority from Japanese patent application No. 2019-184152, filed on Oct. 4, 2019, the disclosure of which is incorporated herein in its entirety by reference.

### REFERENCE SIGNS LIST

1 REHABILITATION PLANNING APPARATUS
2 SIMILARITY CALCULATION UNIT
3 RANKING UNIT
4 DETERMINATION UNIT
10 REHABILITATION PLANNING SYSTEM
20 REHABILITATION PLANNING SYSTEM
30 REHABILITATION PLANNING SYSTEM
100 REHABILITATION PLANNING APPARATUS
101 PAST INFORMATION STORAGE UNIT
102 REHABILITATION HISTORY STORAGE UNIT
103 SIMILARITY CALCULATION UNIT
104 RANKING UNIT
105 OUTPUT CONTROL UNIT
150 NETWORK INTERFACE
151 MEMORY
152 PROCESSOR
200 REHABILITATION PLANNING APPARATUS
201 CLASSIFICATION UNIT
300 REHABILITATION PLANNING APPARATUS
301 HOSPITALIZATION PERIOD ESTIMATION UNIT
302 ABILITY VALUE ESTIMATION UNIT
400 NETWORK
500 TERMINAL DEVICE

What is claimed is:

1. A rehabilitation planning apparatus comprising:
at least one memory storing program instructions; and
at least one processor configured to execute the instructions stored in the memory to:
calculate a degree of similarity between each of a plurality of pieces of past information and target patient information, each of the plurality of pieces of past information being information about a respective one of a plurality of past patients who performed rehabilitation in a past, and the target patient information being information about a target patient;
extract at least one piece of past information from the plurality of pieces of past information based on the degree of similarity, and rank the at least one piece of past information according to a predetermined ranking rule; and
determine, as a rehabilitation plan for the target patient, a rehabilitation history of the past patient associated with the past information according to a result of the ranking of the pieces of past information.

2. The rehabilitation planning apparatus according to claim 1, wherein
each of the pieces of past information and the target patient information includes a history of an ability value for a predetermined rehabilitation period, and
the processor is further configured to execute the instructions to calculate at least a degree of similarity of the history of the ability value.

3. The rehabilitation planning apparatus according to claim 1, wherein
each of the pieces of past information and the target patient information includes a target in rehabilitation, and
the processor is further configured to execute the instructions to calculate at least a degree of similarity of the target.

4. The rehabilitation planning apparatus according to claim 1, wherein
the processor is further configured to execute the instructions to classify the pieces of past information and the target patient information based on information included in the pieces of past information and in the target patient information, and exclude, from information to be processed, information related to pieces of past information that have been classified into a category different from a category in which the target patient information has been classified.

5. The rehabilitation planning apparatus according to claim 4, wherein
each of the pieces of past information and the target patient information includes a history of an ability value for a predetermined rehabilitation period, and
the information used for the classification is the history of the ability value.

6. The rehabilitation planning apparatus according to claim 4, wherein
each of the pieces of past information and the target patient information includes a target in rehabilitation, and
the information used for the classification is the target in the rehabilitation.

7. The rehabilitation planning apparatus according to claim 1, wherein
the past information includes an ability value of the past patient before performing rehabilitation indicated in the rehabilitation history and an ability value thereof after performing the rehabilitation, and
the predetermined ranking rule is sorting based on a degree of improvement in the ability value of the past patient after performing the rehabilitation indicted in the rehabilitation history.

8. The rehabilitation planning apparatus according to claim 7, wherein the processor is further configured to execute the instructions to determine, as a rehabilitation plan for the target patient, a predetermined number of rehabilitation histories of respective past patients associated with respective pieces of past information in descending order of the degree of improvement.

9. The rehabilitation planning apparatus according to claim 7, wherein the processor is further configured to execute the instructions to determine, as examples of failures, predetermined number of rehabilitation histories of respective past patients associated with respective pieces of past information in ascending order of the degree of improvement.

10. The rehabilitation planning apparatus according to claim 1, wherein
when an incident has occurred for the past patient, the past information is associated with incident information indicating the incident that has occurred, and
the processor is further configured to execute the instructions to specify the incident information associated with at least one extracted piece of past information.

11. The rehabilitation planning apparatus according to claim 1, wherein
the past information includes, for each of a plurality of types of abilities, an ability value of the past patient before performing rehabilitation indicated in the rehabilitation history and an ability value thereof after performing the rehabilitation, and
the processor is further configured to execute the instructions to specify a type of ability of which an ability value is improved by performing the rehabilitation indicated in the rehabilitation history of the past patient that has been determined to be the rehabilitation plan for the target patient.

12. The rehabilitation planning apparatus according to claim 1, wherein the processor is further configured to execute the instructions to estimate a hospitalization period under an assumption that the target patient performs the rehabilitation indicated in the rehabilitation history.

13. The rehabilitation planning apparatus according to claim 12, wherein
the past information includes a hospitalization period of the past patient, and
the processor is further configured to execute the instructions to estimate the hospitalization period included in the past information as a hospitalization period that is expected under the assumption that the target patient performs the rehabilitation indicated in the rehabilitation history associated with the past information.

14. The rehabilitation planning apparatus according to claim 12, wherein the processor is further configured to execute the instructions to estimate the hospitalization period by using a prediction model that has undergone a learning process in advance.

15. The rehabilitation planning apparatus according to claim 1, wherein the processor is further configured to execute the instructions to estimate an ability value under an assumption that the target patient has performs the rehabilitation indicated in the rehabilitation history.

16. The rehabilitation planning apparatus according to claim 15, wherein
the past information includes an ability value of the past patient after performing the rehabilitation indicted in the rehabilitation history, and
the processor is further configured to execute the instructions to estimate the ability value included in the past information as an ability value that is expected under the assumption that the target patient performs rehabilitation indicated in the rehabilitation history associated with the past information.

17. The rehabilitation planning apparatus according to claim 15, wherein the processor is further configured to execute the instructions to estimate the ability value by using a prediction model that has undergone a learning process in advance.

18. The rehabilitation planning apparatus according to claim 1, wherein the rehabilitation history includes a time of an occurrence of an outing event of a patient.

19.-24. (canceled)

25. A rehabilitation planning method comprising:
calculating a degree of similarity between each of a plurality of pieces of past information and target patient information, each of the plurality of pieces of past information being information about a respective one of a plurality of past patients who performed rehabilitation in a past, and the target patient information being information about a target patient;
extracting at least one piece of past information from the plurality of pieces of past information based on the degree of similarity, and ranking the at least one piece of past information according to a predetermined ranking rule; and
determining, as a rehabilitation plan for the target patient, a rehabilitation history of the past patient associated with the past information according to a result of the ranking of the pieces of past information.

26. A non-transitory computer readable medium storing a program for causing a computer to perform:
a similarity calculation step of calculating a degree of similarity between each of a plurality of pieces of past information and target patient information, each of the plurality of pieces of past information being information about a respective one of a plurality of past patients who performed rehabilitation in a past, and the target patient information being information about a target patient;
a ranking step of extracting at least one piece of past information from the plurality of pieces of past information based on the degree of similarity, and ranking the at least one piece of past information according to a predetermined ranking rule; and
a determination step of determining, as a rehabilitation plan for the target patient, a rehabilitation history of the past patient associated with the past information according to a result of the ranking of the pieces of past information.
